(19) United States
(12) Patent Application Publication
(10) Pub. No.: US 2015/0355178 A1
(43) Pub. Date: Dec. 10, 2015

(54) SYSTEM AND METHOD FOR IMPROVING BIOMARKER ASSAY

(71) Applicant: MBio Diagnostics, Inc., Boulder, CO (US)

(72) Inventors: Christopher J. Myatt, Boulder, CO (US); Daniel T. Nieuwlandt, Longmont, CO (US); Gregory McLintock Husar, Longmont, CO (US); Charles H. Greef, Louisville, CO (US)

(21) Appl. No.: 14/425,408

(22) PCT Filed: Sep. 4, 2013
(86) PCT No.: PCT/US13/58071

§ 371 Date: Mar. 3, 2015

Related U.S. Application Data
(60) Provisional application No. 61/696,590, filed on Sep. 4, 2012.

(51) Int. Cl. G01N 33/569; G01N 33/53

(52) U.S. Cl. CPC G01N 33/56988; G01N 33/5306; G01N 2469/10

(57) ABSTRACT

The present disclosure pertains to detection of biomarkers in a sample. More particularly, the disclosure relates to methods for treating the sample to liberate certain analytes prior to the assay. Composition for disrupting the HIV virus and antibody-antigen complex to release p24 antigen is also disclosed. The disclosed methods and compositions are compatible with existing HIV antigen/antibody combination assays and improve the sensitivity of such assays.

### RELATED APPLICATIONS

This application claims priority to U.S. Provisional Patent Application No. 61/696,590, filed Sep. 4, 2012, which is incorporated by reference into the present application in its entirety and for all purposes.

### GOVERNMENT INTERESTS

This invention was made with government support under Award Number A1093289 awarded by the National Institutes of Health (“NIH”). The government has certain rights in this invention.

### FIELD OF THE INVENTION

The present disclosure pertains to detection of biomarker(s) in a sample. More particularly, the disclosure relates to compositions and methods for liberating bound analytes prior to an assay.

### BACKGROUND

Early detection of a disease is often critical for successful control and treatment of the disease. Providing accurate, high-speed, and low cost analysis for infection diagnosis, pathogen detection, or other biological or chemical antigen detection remains a major challenge for public health. As a disease progresses through its course, the biomarker profile changes. An assay that can enhance the availability of biomarkers during some or all of the phases of a disease would increase the sensitivity of the test, and thereby improve the control and treatment of the disease. A few examples are disclosed here, and the concept holds in numerous diseases.

At the onset of HIV (human immunodeficiency virus) infection, the virus usually grows slowly at the initial focus of infection, for example, at a mucosal surface. After several days or even weeks, the virus may escape the mucosal tissue into the blood stream and lymphatic system, where it circulates and rapidly propagates in host cells of the blood and lymphatic tissues. This stage of acute viremia is characterized by the appearance and rapid increase of viral biomarkers in the blood. Examples of such viral biomarkers may include nucleic acids and proteins associated with the virus. The exponential growth of the virus is initially limited by the response of the innate immune system of the host. The host immune system is responsible for attacking and destroying foreign objects, such as circulating virions and infected host cells, which may cause further liberation of viral RNA and proteins in the blood. In parallel to the response of the innate immune system, the adaptive immune system begins to respond. For instance, B-cells that are specific for HIV protein antigens may be activated and begin proliferating and generating antibodies against these HIV antigens. As the response of the immune system continues, the blood stream is replete with debris resulting from dead cells or lysed viruses. Immunogenic antigens are often complexed with antibodies. In the absence of treatment, the battle continues for a number of years until the immune system is exhausted. The virus once again proliferates, destroying the remainder of the host immune cells. The health of the patient deteriorates, leading to Acquired Immunodeficiency Syndrome (“AIDS”).

HIV tests for detecting host antibodies against HIV have been developed and these tests are useful in diagnosing most of the cases after the initial infection and viremia. An improvement to this assay format combines antibody detection with direct detection of HIV proteins. It is during early infection that diagnostically useful HIV viral capsid p24 protein becomes readily detectable by sensitive immunoassays. Following subsequent immune responses to HIV antigens, however, the p24 antigen (and other HIV antigens) becomes predominantly bound to specific antibodies and is no longer demonstrably detectable by conventional immunoassays. The combination of simultaneous antigen and antibody detection, a so-called “Fourth generation HIV test,” provides improved performance for an immunoassay diagnostic test. Early detection of p24 antigen is often hampered by incomplete immune responses at the early (“acute”) stage of fighting HIV. In these cases, many immunogenic antigens are bound up in intact viral particles, infected cells, immune complexes, and other structures where they are not accessible by conventional immunoassay. At a later time during the course of the disease, the destructive power of the immune systems liberates these antigens. However, earlier detection of the disease may enable prompt isolation of the patient and/or more effective treatment.

The performance of an HIV assay is measured, at least in part, by how early the assay can detect infection. Substantial resources have been invested in developing HIV screening and diagnostic techniques with the aim of shortening the time between initial infection and detection of the disease. This time gap, between the moment of infection and the time at which analytes are available in sufficient quantity to be detected by a given assay, is called the window period—a period during which a given assay technique cannot detect the presence of infection. Currently, the technique with the shortest window period for HIV is reverse transcription-polymerase chain reaction (“RT-PCR”) amplification of viral RNA. However, this technique is a laborious and expensive laboratory-based technique. A lower cost, more practical approach is immunoassay. The progression of HIV immunoassay technology improvements, from crude viral lysate immunoassay to sandwich immunoassay to combination antibody/antigen immunoassay, has significantly shortened the window period for this most popular and affordable screening tool.

Current combination antibody/antigen immunoassays, for example, fourth generation HIV tests that combine the detection of antibodies with the detection of circulating viral proteins, have a window period that is several days longer than that of RT-PCR. These fourth generation HIV tests typically detect the viral capsid protein p24, which is a structural protein that forms the capsid underneath the viral membrane. A key limitation of these assays during early infection is that the capsid protein cannot be captured in the immunoassay when the virion is intact, or if the capsid protein is bound in other immune-complexes. In fact, free p24 protein only appears in the blood stream after the exponential expansion of virus, when the immune system begins to mount a significant response, which is typically days after the onset of viremia. See Karris et al. (2012).

In general, many biomarkers are bound by immune system components or in structures such as intact virions, intact bacteria, or other organisms. Such biomarkers include, for example, viral particles and proteins, bacteria and bacterial antigens, self-reactive antigens in autoimmune disease, and other immune system targets. Immune complexes are found in two main “compartments” of the circulatory system, (1) freely floating in the plasma as circulating immune complexes (CICs), and (2) bound immune complexes (BICs) bound to receptors of the circulatory cells, such as to erythrocytes. The specific detection of analytes sequestered within immune complexes allows the earlier detection of disease and/or the detection of disease that is characterized by slow progression. The term “IC” refers to immune complexes, which may include CICs and BICs.

In the case of tuberculosis (TB), Mycobacterium tuberculosis (MTB) is a slow growing bacterium that can occur as a latent infection wherein the organisms are encapsulated by the immune system, for example, as nodules in the lungs, or as active disease characterized by ongoing replication and battle with the immune system. In managing TB disease, it is critical to quickly understand whether an active infection is indeed occurring. In either latent infection or active infection, the immune response includes a mature antibody response, since any clinically relevant symptoms occur weeks after the onset of active disease. See Brighenti, S., and Lerm, M (2012). How Mycobacterium tuberculosis Manipulates Innate and Adaptive Immunity—New Views of an Old Topic, Understanding Tuberculosis—Analyzing the Origin of Mycobacterium Tuberculosis Pathogenicity, Dr. Pere-Joan Cardona (Ed.), ISBN: 978-953-307-942-4. See also, Vankayalapati, R., Barnes, P., (2009), Innate and adaptive immune responses to human Mycobacterium tuberculosis infection. Tuberculosis 89, 51, 577-580. Thus any antigens either secreted by the growing MTB infection, or antigens resulting from the battle with the immune system, would be quickly opsonized by either (1) complement through the innate response, or (2) antibody plus complement through the adaptive response. The resulting immune complexes may be attached to blood cells such as erythrocytes through complement receptors. Thus the level of un-complexed antigen in serum or plasma is expected to be very low, while detectable levels occur in immune complexes. In fact, circulating immune complexes (CICs) have been studied for TB disease, albeit in a non-specific manner of quantifying the precipitate of CICs. Studies on the quantity of CICs show a dramatic rise during late-stage TB disease, highly suggestive that CICs contain TB antigen (see Arora A, et al 1991).

BICs bound to erythrocytes may also include TB antigen, and are expected to be present earlier in the infection than CICs—in short, significant quantities of CICs likely appear only after the available binding sites on erythrocytes and other cells have been saturated. Thus a method for liberating BICs, and specifically assaying the antigens therein would enable an earlier, and highly specific, test for the condition of active TB disease.

There are a wide variety of diseases where the availability of antigen is reduced due to sequestration in an immune complex or in an intact structure such as a virion. Chronic disease such as hepatitis B and C would also have significant quantity of immune complexes during the chronic phase. A test that provides an increased bio-availability of sequestered markers, whether combined with specific host antibody detection or not, would provide greater diagnostic sensitivity.

### SUMMARY

The present disclosure advances the art by providing a system and method for enhanced detection of biomarkers, such as pathogen proteins. In one embodiment, one approach for early detection of the HIV capsid protein is to lyse the virion prior to immunoassay in order to liberate the capsid protein. In another embodiment, methods are disclosed for improved detection of biomarkers that are bound by immune system components. Such biomarkers may include but are not limited to viral particles, viral proteins or other antigens, bacteria and bacterial antigens, self-reactive antigens in autoimmune disease, or other immune system targets.

One of the most prominent biomarkers for HIV is the p24 antigen (also referred to as p24 or p24 protein). p24 is an HIV viral core (capsid) protein and is the most abundant HIV viral protein with over 1,000 molecules per virion (See Layne et al. (1992) and Summers et al. (1992). The levels of p24 in host blood increase over time after infection of the host by HIV. However, the sensitivity of conventional immunoassays is not high enough to detect p24 in the blood at the early stage of HIV infection when p24 levels are relatively low. Current p24 immunoassays compromise sensitivity for practicality.

Not all p24 proteins in a sample are extraviral. p24 proteins that are associated with intact viruses are usually not detectable. Moreover, in seroconverted individuals, extraviral p24 is predominantly immunocomplexed and generally unavailable for capture in p24 immunoassays. To improve the sensitivity of p24 assays, samples may be subject to treatment by detergents and heat, or by acid followed by neutralization, to release p24 from both viral particles and anti-p24 antibodies. See e.g., Schupbach et al. (2006); Nishanian et al. (1990); and Schupbach et al. (1996). For example, the commercial p24 ELISA kit from PerkinElmer® uses a detergent and neutralization approach for immune complex disruption. Parpia et al. (2010) describe a method in which heat shock is used to improve p24 antigen detection sensitivity in a rapid test format. Methods that use chemical or heat decomplexation, however, can lead to denaturation of sample antibodies, compromising the ability to detect both antigen and antibody in a sample. For example, decomplexation methods applied to blood, serum, or plasma from HIV-infected individuals may compromise the antibody detection aspect of the fourth-generation assay, or associated antibody detection based co-infection serology assays. In an embodiment, the present disclosure provides a method for disrupting the viruses which helps increase the detectable concentration of p24 without significantly compromising the ability of a fourth generation assay to also detect anti-HIV antibodies.

Virions may be disrupted to release RNA or proteins from within the virion. Techniques for disruption may include but are not limited to heat, sonication, and chemical lysis. In one aspect, heat may be used to liberate bound p24 antigen from HIV. However, some of these disruption techniques may result in the denaturation of sample antibodies, rendering the sample not amenable to the serology component of fourth-generation HIV assays.

Disruption of HIV virus using non-ionic detergents alone is suboptimal. It has been demonstrated that a combination of certain detergents together with heat (10 minutes at 70° C.) significantly improves p24 release. See Schupbach (2006). However, these conditions may denature sample antibodies under certain conditions.

In one embodiment, this disclosure provides a unified assay that uses a virus-disrupting composition to disrupt the viruses in order to enhance p24 detection during the earliest stages of viremia. The disclosed virus-disrupting composition is sufficiently mild such that it does not significantly interfere with detection of antibodies in the same sample. Thus, the disclosed method and composition for early detection of viral antigens may be combined with antibody detection, to provide a comprehensive detection format over all stages of HIV disease.

It has been reported that the erythrocyte fraction of a whole blood sample may also be a source of HIV antigens and RNA. See Steinmetzer et al. (2010) and Garcia et al. (2012). Evidence suggests that some p24 antigen is adsorbed onto the membranes of erythrocytes. In laboratory-based 4^{th} generation tests, the sample is serum or plasma (for example, Abbott ARCHITECT HIV Ag/Ab Combo, and the Bio-Rad GS HIV Ag/Ab Combo), and thus cannot detect erythrocyte bound p24 antigen. Other assays that use whole blood, such as lateral flow assays, typically incorporate membrane filtration/removal of erythrocytes prior to disruption, if disruption is used at all. See Nabitayan A (2011). In one embodiment of the present disclosure, the use of whole blood sample may enhance the detection of p24 antigen. In another embodiment, the application of viral lysis in whole blood sample may further increase the sensitivity of the assay. In yet another embodiment, additional assay components that specifically disrupt complement may also be included.

The disclosed assays provide a valuable improvement over the fourth generation HIV assays because current fourth generation assays can only detect free viral antigen and host antibodies. Moreover, the disclosed methods, when used in conjunction with an appropriate platform, provide more sensitive assays than current assays available on the market.

In one aspect, the disclosed early detection of antigen and antibody may be combined with an assay platform suitable for point of care (“POC”) operation. Examples of such a platform and related methods are described in International Patent Application PCT/US2011/051791 entitled “SYSTEM AND METHOD FOR DETECTING MULTIPLE MOLECULES IN ONE ASSAY,” and in U.S. patent application Ser. No. 13/831,788 entitled “SYSTEM AND METHOD FOR DETECTING MULTIPLE MOLECULES IN ONE ASSAY,” both of which are hereby incorporated by reference into this disclosure in their entirety.

In another aspect, a method for determining the level of one or more biomarkers in a sample is provided, which may include, among others, the following steps: (a) contacting the sample with a composition to form a sample mixture, wherein the composition comprises an ionic detergent, a nonionic detergent, and a salt; (b) loading the sample mixture into a device comprising a waveguide, allowing the one or more biomarkers to bind to one or more capture molecules immobilized on the waveguide; (c) adding one or more labeling molecules into the device, allowing the labeling molecules to bind to their respective biomarkers, and (d) measuring the signal intensity emitted from the labeling molecules that are bound to the immobilized biomarkers and capture molecules on the waveguide to determine the level of the one or more biomarkers in the sample. Examples of the sample may include but are not limited to whole blood sample, serum, plasma or saliva. The disclosed method may further include a heating step wherein the temperature of the sample mixture is raised to at least 70° C., 80° C. or 90° C. after step (a) but before the sample mixture is loaded into the device in step (b).

For purpose of this disclosure, “determining the level of one or more biomarkers” may include measuring the counts or concentrations of one or more biomarkers qualitatively, quantitatively, or semi-quantitatively, and may also include determining the total counts of a pathogen (e.g., viruses or bacteria) in a sample in a qualitative, quantitative, or semi-quantitative manner. A qualitative assay typically provides a Yes or No answer with respect to the presence/absence of a particular biomarker, whereas a quantitative or semi-quantitative assay provides a more specific count or concentration of the biomarker.

In another aspect, the sample may contain a plurality of (i.e., more than one) biomarkers which may include, for example, at least one antigen originated from a pathogen and at least one antibody against the pathogen. The waveguide-based device may contain a plurality of capture molecules, where at least one group of capture molecules is capable of capturing the at least one antigen, while at least one other group of capture molecules is capable of capturing the at least one antibody.

In another aspect, the composition may have a pH of lower than 3.5, or lower than 2.5, to help disrupt the immune complex containing the target biomarker(s). When such an acidic composition is added to the sample, a neutralizing solution may be needed after step (a) but before step (b) to neutralize the sample mixture pH before loading.

The combination of the disclosed lysis composition with the waveguide based technology may be particularly valuable because it provides fast and accurate assay at the point of care, while being sufficiently inexpensive to permit wide screening of a population. For instance, whole blood sample may be directly used for the assay without removal of RBCs. In one aspect, using the disclosed composition and methodology, the assay may be capable of producing a statistically significant positive signals from a sample having an HIV viral load of 1,000, 2,000, 2,500, 5,000, 10,000, 50,000, 100,000, 200,000, 300,000, 400,000 copies/ml or lower.

In another embodiment, the composition may contain an ionic detergent, a nonionic detergent, a salt, and one or more labeling molecules that bind to the biomarkers. The biomarkers may bind to the labeling molecules at the same time when the sample is treated with the lysis buffer (e.g., VDSA). In another embodiment, the one or more labeling molecules may be embedded in the waveguide based device. The treated sample may get in contact with the labeling molecules after being loaded into the device.

Besides HIV, the disclosed methods may be applied to detect other diseases, such as tuberculosis, viral hepatitis, and so forth. The techniques described here may allow for enhanced detection of bound antigen, and may also enable detection of host antibody response at the same time. Those skilled in the art would understand the applicability of the embodiments to the detection of biomarkers to other diseases.

### BRIEF DESCRIPTION OF THE DRAWINGS

FIG. 1 shows detection of anti-gp41 antibody present in HIV-1 positive samples treated with “virus disruption sample additive” (VDSA) as compared to samples treated with conventional methods.

FIG. 2 shows detection of p24 antigen in samples comprised of non-complexed p24 antigen spiked into normal human serum. Samples treated with VDSA as compared to samples treated with conventional methods.

### DETAILED DESCRIPTION

Methods for improving antigen detection are disclosed. More specifically, test samples may be treated to liberate certain analytes prior to the assay. Composition is disclosed for disrupting HIV viruses to release p24 antigen. The disclosed methods and composition are compatible with existing HIV antigen/antibody combination assays and improve the sensitivity of such assays. In one aspect, the disclosed methods and composition help release more HIV antigen (e.g., p24) into the sample enabling more sensitive detection of such antigens at an early stage of HIV infection. In another aspect, the disclosed composition does not significantly interfere with antibodies present in the sample. Thus, the disclosed methods and composition enable simultaneous detection of HIV antigen(s) and host antibodies against HIV antigens.

In one embodiment, HIV p24 antigen may be detected at an early stage post infection. In one aspect, p24 antigen may be detected as early as one day, two days, four days, or one week after infection of the host by HIV. In another aspect, p24 antigen may be detected in a sample having an HIV viral load of 1,000, 2,000, 2,500, 5,000, 10,000, 50,000, 100,000, 200,000, 300,000, 400,000 copies/ml or lower.

In one aspect, the virus-disrupting composition, also referred to as “virus disruption sample additive” (VDSA), may contain at least one non-denaturing (non-ionic) detergent, for example, Triton X-100, and at least one denaturing (ionic) detergent, for example, deoxycholate. The VDSA may further contain a salt. The VDSA is capable of enhancing release of p24 antigen from the HIV virus in the test sample while maintaining the ability to detect host antibodies against HIV antigens in the same sample. In another aspect, VDSA may also contain one or more zwitterionic detergents. The compositions disclosed herein may be capable of disrupting virus holding target biomarkers, or disrupting immune-complex containing target biomarkers, or some compositions may be capable of performing both functionalities.

In one embodiment, the VDSA may have a formula as follows: 2.5% v/v Triton® X-100, 2.5% w/v sodium deoxycholate, 0.5% w/v sodium dodecyl sulfate (SDS), 750 mM NaCl, 17 mM EDTA, and 50 mM Tris-CI, pH 7.4. In another embodiment, the VDSA may have a formula as follows: about 2.5% v/v Triton® X-100, about 2.5% w/v sodium deoxycholate, about 0.5% w/v sodium dodecyl sulfate (SDS), about 750 mM NaCl, about 17 mM EDTA, and about 50 mM Tris-CI, pH 7.4.

In another embodiment, the non-ionic detergent may be one or more detergent selected from the group consisting of Triton® X-100, Triton® X-114, Brij®-35, Brij®-58, Tween® 20, Tween® 80, and NP-40. In one aspect, the VDSA may contain Triton® X-100 at a concentration of 1-5% v/v. In another aspect, the non-ionic detergent may be Triton® X-100 at a concentration of 2-3% v/v.

In another embodiment, ionic detergent may be one or more detergent selected from the group consisting of deoxycholate, SDS, sodium glycocholate, and hexadecyltrimethylammonium bromide (CTAB). Examples of deoxycholate may include but are not limited to sodium deoxycholate. In another aspect, the VDSA may contain sodium deoxycholate at a concentration of 1-5% w/v. In another aspect, the VDSA may contain sodium deoxycholate at a concentration of 2-3% w/v. In another aspect, the VDSA may contain SDS at a concentration of 0.1-1.5% w/v. In another aspect, the VDSA may contain SDS at a concentration of 0.2-1% w/v.

VDSA may also include betaine derivatives. One example of such a derivative is Empigen BB (N,N-Dimethyl-N-dodecylglycine betaine, Sigma #45165).

In one aspect, the VDSA may contain NaCl at a concentration of 100 mM to 1M. In another aspect, the VDSA may contain EDTA at a concentration of 5-50 mM. In another aspect, the VDSA may contain Tris-CI at a concentration of 10-200 mM. In another aspect, the VDSA may have a pH in the range of 6.5-8. In another aspect, the VDSA may also contain an anti-CD59 antibody.

In one aspect, the disclosed composition may be mixed with the sample at a certain ratio to form a sample mixture, wherein the concentration of the SDS in the sample mixture is in the range of 0.01-0.3% (w/v). In another aspect, the concentration of the sodium deoxycholate in the sample mixture may be in the range of 0.1-1% (w/v) after the disclosed composition is mixed with the sample at a certain ratio. In another aspect, the concentration of the Triton® X-100 in the sample mixture may be in the range of 0.1-1% (v/v) after the disclosed composition is mixed with the sample at a certain ratio. By way of example, the VDSA may be added to the test sample at a ratio of about 1:2 (v/v) to about 1:8 (v/v) between VDSA and test sample. Remaining final assay sample volume may be made up of water, suitable buffers or sample dilution buffer. Sample dilution buffer may contain detection agents, such as anti-p24 antibody and HIV antigen, among others.

In another embodiment, acids or other reagents may be used to dissociate p24 antigen from erythrocytes in a sample, such a whole blood sample. By way of example, a solution of glycine-HCl (e.g. glycine-HCl buffer of pH 3.2, see e.g., Garcia 2012) may be mixed with the sample, followed by neutralization with a base (such as NaOH) to about pH 7.2. The cellular components in the sample may or may not be separated during the acid-mixing and neutralization steps. This acid dissociation step may be performed independently or it may be combined with viral lysis steps disclosed herein or with other viral lysis techniques well known in the art.

A further benefit of an assay that specifically detects antigen within both BICs and CICs is that it enables monitoring of the efficacy and progression of a course of therapy for TB. Upon the application of an effective therapy, the number of shed antigens should increase. In fact, it has been observed that the quantity of CICs is higher in acute and chronic infections than in non-infected samples at the start of TB chemotherapy, increases markedly at the beginning of therapy, then later decreases to levels below pretreatment. See Raja, A., Ranganathan, U., Bethunaicken, R., (2006), Clinical value of specific detection of immune complex-bound antibodies in pulmonary tuberculosis. Diagnostic Microbiology and Infectious Disease, 56: 281-287. See also, Samuel, A., Ashtekar, M., Ganatra, R., (1984) Significance of circulating immune complexes in pulmonary tuberculosis. Clin. Exp. Immunol. 58; 317-324; and Johnson, N., McNicol, M., Burton-Kee, E., Mowbray, J. (1981) Circulating Immune Complexes in tuberculosis. Thorax, 36:610-617. This monitoring enables confirmation of the TB diagnosis, as well as a test of drug susceptibility of the particular strain infecting the patient. Thus an assay that is sensitive to CICs and BICs would have significant clinical importance in therapy monitoring. In general, this approach to therapy monitoring may be useful for many types of infections.

In another embodiment, the disclosed methods may be useful in situations where ICs are generated in a disease that occurs in a body compartment where sample collection is difficult, invasive, or inconvenient. One such example is in influenza detection, where some level of antigen is expected in the circulatory system due to the apoptosis of epithelial cells that contain viral particles, and the removal of those apoptotic cells and viral debris by the circulatory system. Antibody response usually exists within influenza patients due to past infections or vaccinations. These antibody responses are not specific enough to prevent active infection by the virus, but can lead to opsonization and IC formation. These ICs can then be detected in the serum, plasma, and bound to erythrocytes and other cells. Given the low level of antigenemia of a nasal infection, any ICs would quickly bind to cell receptors, leading to BICs. A method that liberates the BICs, concentrates the BICs and CICs, then de-complex the BICs and/or CICs, and finally specifically detect the previously complexed biomarkers, may prove useful in diagnosing respiratory infections using a blood sample. The biomarkers may be proteins, nucleic acids or other biological molecules. The quantitative monitoring of specific biomarkers in CICs and BICs may be valuable for monitoring the efficacy of a particular therapy.

Other examples of disease where sample collection is difficult, invasive, or inconvenient include infections of the uro-genital tract, such as gonorrhea and chlamydia; and infections of the central nervous system, such as meningitis. The quantitative monitoring of specific biomarkers in CICs and BICs may prove valuable for detection of disease as well as in monitoring of therapy.

Another example of the application of the disclosed methods is in an antigen assay using a nasal swab sample. Because IgA antibodies are released into the mucus, opsonization of respiratory pathogen antigen, as well as bacterial and virus particles, may occur in a mucus sample. Generally, any sample from any mucosal surface may contain opsonized or sequestered biomarkers. Breaking up immune complexes prior to an antigen assay from a swab sample may lead to enhanced sensitivity.

Another disease where BICs and CICs may contain the majority of available biomarkers in a blood sample is sepsis. Since many of the bacteria that are suspected in sepsis are common bacteria, such as staphylococcus, the patient likely has antibodies to the infectious agent. Thus any antigen or whole organisms would be opsonized and confined to the BICs and CICs, particularly early in the infection. Liberating BICs and breaking up immune complexes prior to an assay from the sample may lead to enhanced sensitivity.

In another embodiment, BICs and CICs may play a role in diagnosis of cancer. If a cancer correlates with a mutated protein, that protein may be antigenic and an adaptive host response may be formed against this antigen. Opsonization of that antigen may lead to CICs and BICs. An assay sensitive to the biomarker content of these ICs may be used as a diagnostic for cancer. Further, any effective chemotherapy may generate an increase of these ICs. Thus a quantitative assay for the contents of ICs may provide a useful tool for therapy monitoring as well as for cancer detection.

In another embodiment, BICs and CICs may play a role is in autoimmune disease. By definition, some self-antigen is being targeted by the immune system, and the presence of ICs which also contain auto-antibodies could be a useful diagnostic for autoimmune disease. Note that in this case, the important biomarker is the auto-antibody, rather than the antigen. Presumably the antigen is available in any host sample; if not, then detection of the antigen may be useful.

The quantitative measure of the contents of CICs and BICs, and its use for therapy monitoring, may be useful in monitoring therapeutic effectiveness for a number of diseases. Examples include hepatitis C and HIV, where effective therapy should lead initially to a rapid rise in antigen, then a subsequent decline.

In another embodiment, it may be useful to separately detect free antigen in the plasma and antigen bound in CICs or BICs. An assay may be set up with two read-out sections. In one section, only antigen that is freely available in plasma or serum is detected. In the other, antigen bound in ICs is detected. An exemplary use of such an assay is in monitoring therapy for HIV by monitoring the presence of p24 antigen. The presence of antigen in serum and plasma—both free antigen and CICs—indicates the most recent virologic replication, while that in BICs indicates the level of replication integrated over the recent past, approximately over several weeks of time. The average lifetime of a BIC is a combination of the rate for removal of BICs from erythrocytes, convolved with the lifetime of the erythrocytes.

Another potential application of the disclosed methods is in malaria testing, where low levels of antigen in BICs may indicate sub-clinical infection, while the presence of free antigen and CICs is indicative of clinical manifestation.

The separate detection of CICs and free antigen from that in BICs may also allow better interpretation during therapy monitoring. Shortly after the initiation of an effective therapy, one would expect a rapid rise of free antigen and CICs, followed by a sharp decline to zero. However BICs would rise along with the rise of the free antigen and CICs, but then reach a plateau as the free antigen and CICs decline. The level of the plateau would then slowly decline over the time scales for elimination of BICs from the erythrocytes, convolved with the time scale for death of the erythrocytes.

In another embodiment, a system may be designed where free antigen, CICs, and BICs are separated and analyzed separately. A number of techniques may be employed to achieve such separation, either into three separate pools, or into two classes, namely, (1) free antigen and CICs; and (2) BICs. An example is to spin down a tube of blood, and drawing off the plasma that would yield free antigen and CICs. The erythrocyte fraction may then be separated, acid washed to liberate BICs, and then the BICs separately analyzed. Alternatively, the acid wash for liberating BICs may be added before centrifugation, followed by neutralization. This alternate technique will not distinguish between signals from BICs and those from CICs.

Another method to enhance sensitivity of biomarker detection assays when considering detection of antigens associated with viral or bacterial infections is to cause disruption of viral or bacterial particles in a patient sample, releasing antigens from within these particles, making them available for capture by an analytical device. Techniques for increasing release of the biomarkers from these particles are provided in this disclosure. For example, certain anionic, cationic, zwitterionic, or non-ionic detergents are known to cause disruption of intact viral or bacterial cells, which have been described in previous sections.

In certain infectious disease situations, the infectious agent parasitizes host cells, so that the majority of infectious disease agent biomarker material is contained within the host cells. Thus, a sample pretreatment method that ruptures biomarker-containing host cells may be employed to release sequestered biomarkers and to increase their concentration, enabling greater sensitivity of a biomarker detection assay. For instance, the malaria-causing parasite Plasmodium falciparum infects hosts by invading red blood cells, and during most of their life cycle are sequestered within red blood cells. A pretreatment step in which red blood cells containing malaria biomarkers are ruptured may increase the concentration of the biomarkers for detection.

In another embodiment, a sample treatment that causes both the disruption of ICs and the disruption of viral or bacterial cell or particle may be used to further enhance the sensitivity of the assay. For instance, in the early phase of HIV infection, circulating viral particles contain antigens such as p24 protein, while a nascent immune response can target any or all p24 protein that is freely circulating and bind it into a CIC. As a result, very little free p24 proteins are available for detection. By subjecting a sample to a pretreatment process that disrupts both the ICs and virus particles, a maximal amount of p24 protein would be released and made available for detection by a suitable biomarker detection device.

In some cases, some BICs may be associated with red blood cells. Removal of red blood cells from a patient sample containing such BICs diminishes the measurable quantity of analyte. In this case, subjecting an intact whole blood sample, rather than serum or plasma sample to a pretreatment step meant to disrupt the ICs may yield higher quantities of analyte available for detection, enabling a more sensitive detection assay.

Similarly, in cases where viruses or other infectious agents are associated with red blood cells, it may be advantageous to assay for an analyte directly from whole blood, in which viral particles bound to or contained within red blood cells are disrupted.

Many assay methods, such as ELISA, EIA, or lateral flow, cannot use whole blood as the sample matrix. Certain components of whole blood, such as red blood cells, cause high levels of interference in these assay platforms, compromising their usefulness. Therefore, an assay platform for which whole blood as a sample matrix is acceptable may enable both simpler and more effective detection and diagnostic methodologies.

A waveguide based sensor, in which analytes are detected at the surface of the detection device provides a platform that would be insensitive to the compromising effects of whole blood as sample matrix. Therefore, using a waveguide based biosensor for detection of analytes from whole blood samples, using techniques to disrupt complexes of bound analyte and antibodies or to release analyte from viral particles represents a novel methodology towards improving the field of biomarker detection and infectious disease diagnosis. More details of the waveguide-based devices and methods of their use are disclosed in U.S. patent application Ser. No. 13/233,794, which is hereby incorporated by reference into this disclosure.

In another embodiment, methods for detecting the contents of immune complexes are disclosed. More specifically, test samples may be treated to liberate certain analytes prior to an assay. Compositions and methods are disclosed for disrupting immune complexes to release biomarkers. The disclosed methods and compositions are compatible with existing assays and improve the sensitivity of such assays, particularly for early detection of diseases. In one aspect, the disclosed methods and compositions help release more biomarkers into the sample enabling more sensitive detection of such biomarkers at an early stage of disease. In another aspect, some of the disclosed methods and compositions enable simultaneous detection of antigens and host antibodies.

Although the disclosed method and composition are suitable to be combined with HIV detection assays, it is to be understood that the composition may be used in other settings to release target analytes in a test sample. It is also to be noted that the system and method disclosed herein may be combined with the system disclosed in International Patent Application PCT/US2011/051791 or with any other suitable assays and platforms for detection of biomarkers such as the Alere Determine™ HIV-1/2 Ag/Ab Combo, Abbott ARCHITECT HIV Ag/Ab Combo, and the Bio-Rad GS HIV Ag/Ab Combo, among others.

The following examples are provided for purposes of illustration of embodiments only and are not intended to be limiting. The reagents, chemicals and instruments are presented as exemplary components or reagents, and various modifications may be made in view of the foregoing discussion within the scope of this disclosure. Unless otherwise specified in this disclosure, components, reagents, protocol, and other methods used in the system and the assays, as described in the Examples, are for the purpose of illustration only.

### Example 1

### Effects of Non-Ionic and Ionic Detergents on Viral Lysis and p24 Antigen Detection

In order to improve the detection limit of p24 antigen, non-denaturing (non-ionic) detergents were evaluated alone or in combination with denaturing detergents for effectiveness at improving p24 antigen detection while maintaining the ability to detect sample antibodies. One such detergent formula (also referred to as “virus disruption sample additive” (VDSA)) is as follows: 2.5% v/v Triton X-100, 2.5% w/v sodium deoxycholate, 0.5% w/v sodium dodecyl sulfate (SDS), 750 mM NaCl, 17 mM EDTA, and 50 mM Tris-CI, pH 7.4. This additive may be added to the test sample so that the additive makes up about 14% of assembled assay sample volume. Thus, the assembled assay sample volume would contain, by volume, 56% sample, 14% VDSA, and 30% sample dilution buffer containing biotinylated anti-p24 antibody and HIV antigen detection reagents [1× phosphate-buffered saline, pH 7.4 (Fisher Bioreagents #BP399-1), 0.67 mg/ml mouse IgG (Roche Custom Biotech, Indianapolis, Ind. #11200941103), 1.33 mg/ml poly-mouse IgG (Roche Custom Biotech #11939661103), 0.33% Tween-20, 10 mg/ml bovine serum albumin (BSA), 2 mg/ml poly-BSA Type II (Roche Custom Biotech #11816438103), 0.025% sodium azide, 4 nM biotin-gp41 HIV antigen (Fitzgerald Industries International #30-AH26), and 73.3 nM anti-p24 monoclonal antibody (US Biological #H6003-30A)]). According to the volume percentage of the assembled assay sample volume described above, VDSA contributed the following to the final assembled sample reaction volume: 0.35% v/v Triton X-100, 0.35% w/v sodium deoxycholate, 0.07% w/v sodium dodecyl sulfate (SDS), 105 mM NaCl, 2.38 mM EDTA, and 7 mM Tris-CI, pH 7.4

Assays were initiated by combining 19 μl sample (whole blood, plasma or serum) with 4.8 μl VDSA and mixing via simple aspiration. Following a 10-minute room-temperature incubation, 10.2 μl of sample dilution buffer was added and the assembled assay reaction volume was mixed and the mixture was added to the entrance port of an HIV Ag/Ab combo assay waveguide cartridge. Non-VDSA control sample reactions were comprised of 21 μl sample and 9 μl sample dilution buffer. Anti-p24 monoclonal antibody (“capture mAb”), HIV-1 antigen gp41, and control features were printed in a spatial array on the waveguide. During a 20-minute incubation period, p24 was complexed by the biotinylated detect mAb and the immobilized capture mAb, which facilitates detection of p24 antigen in the sample. In the meantime, anti-gp41 antibodies present in the sample bridged biotinylated detect gp41 and immobilized capture gp41 which facilitates detection of anti-gp41 antibodies in the sample. Following this incubation period, detection of these immobilized biotinylated complexes was achieved by adding 80 μl of 3 nM streptavidin-SureLight P3 conjugates (SA-SLP3), with incubation for an additional 15 minutes at room temperature. Following a 200-μl wash with 200 mM NaCl, 2 mg/ml BSA, 0.2% v/v Tween-20, and 1×PBS, pH 7.4, waveguides were imaged on a fluorescence reader to analyze light signals emitted by the different printed capture agent spots on the cartridge. More details of the waveguide based device and its use are described in U.S. patent application Ser. No. 13/233,794, which is hereby incorporated by reference into this disclosure.

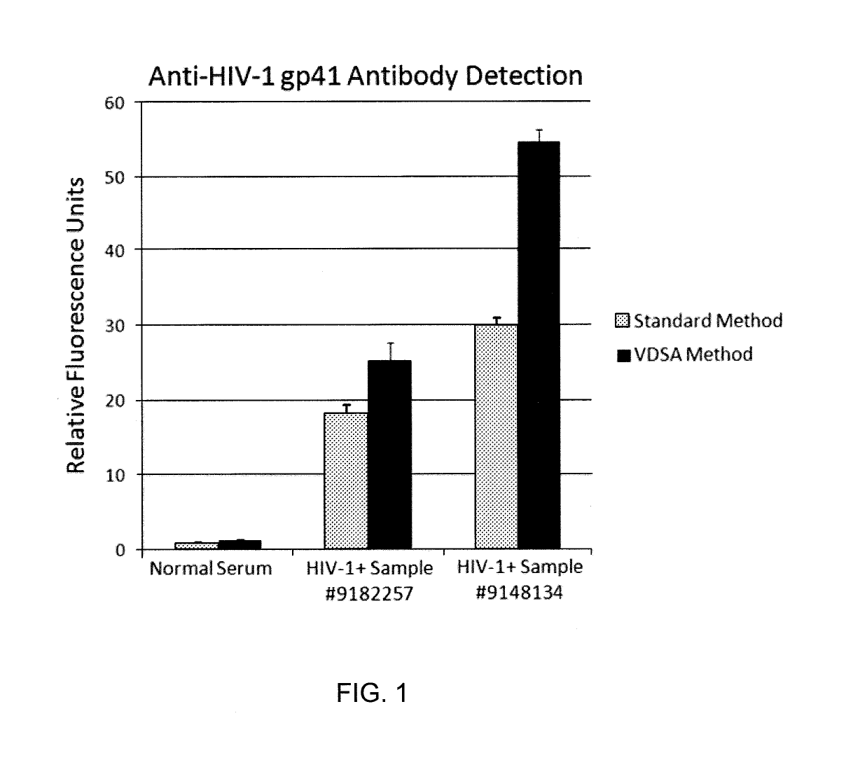

As demonstrated here, VDSA does not eliminate the detection of anti-gp41 sample antibodies in the serology component of the fourth-generation HIV-1 Ag/Ab combo assay. Both the VDSA and non-VDSA protocols described above were applied to a normal serum sample (in duplicate) and two different seroconverted HIV-1 positive control samples (SeraCare #9148134 and SeraCare #9182257) that were pre-diluted 20-fold into normal serum. The dilution of the positive control samples was intended to provide more challenging anti-gp41 antibody titers. The assay signal results, shown in FIG. 1, indicate that anti-gp41 antibody present in the HIV-1 positive samples is not adversely affected by treatment with VDSA.

### Example 2

### VDSA does not Adversely Affect the Activities of the Immunoassay

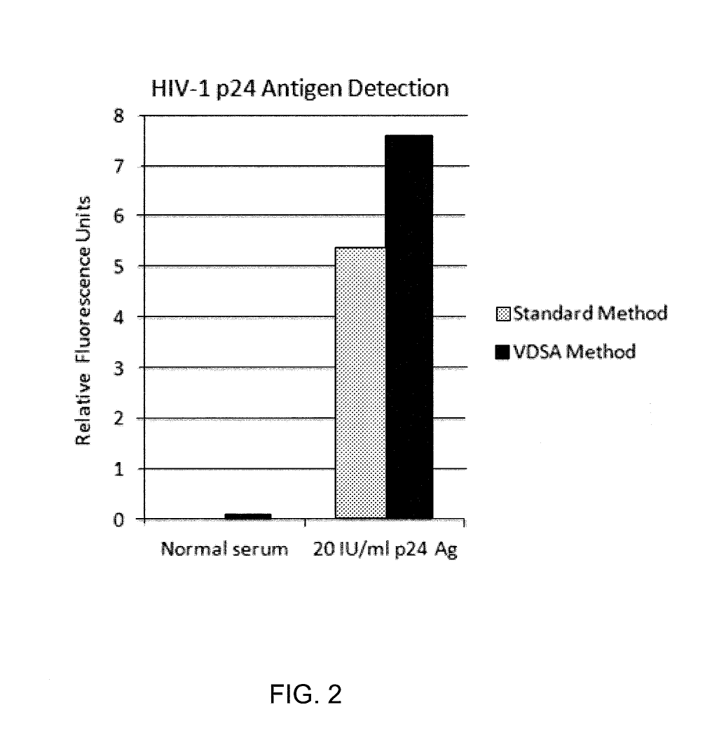

It was additionally demonstrated that VDSA does not adversely affect the activities of the immunoassay using anti-p24 antibodies (capture and detect antibodies). The negative control for this assay was normal serum, while the positive sample was 20 IU/ml WHO International standard HIV-1 p24 antigen [National institute for Biological Standards and Control (NIBSC) code 90/636; Potters Bar Hertfordshire, U.K.). The protocol described above in Example 1, in the presence or absence of VDSA, was applied to these samples. The results are shown in FIG. 2. It is important to note that this standard p24 antigen was prepared by detergent treatment of HIV-1 positive serum and is assumed to be extraviral; therefore, VDSA was not expected to significantly enhance p24 Ag detection.

### Example 3

### Effect of VDSA Protocol on Detection of p24 Antigen in Acute HIV Infection Samples

To demonstrate the effect of the VDSA assay protocol on the detection of p24 antigen in acute HIV infection samples, five plasma samples that are HIV RNA-positive but EIA- and Western blot-negative were assayed with the standard and VDSA Ag/Ab combo protocols as described in Example 1. For comparative purposes, this sample set was also assayed with the Alere Determine™ HIV 1/2 Ag/Ab Combo assay by following the protocol in the product insert that was commercially available from Alere Ltd. (Stockport, United Kingdom). The “Standard Method” is the method of EXAMPLE 1 without the VDSA step. The “VDSA Method” is as described in EXAMPLE 1. The signal-to-cutoff (s/co) data tabulated below demonstrates that the VDSA protocol step significantly increases the concentration of detectable p24 antigen and that the performance of the antigen detection component of the combo assays compares well to that of the commercial Alere Determine™ assay.

TABLE 1
Tests to Compare Sensitivity of Different Assays

| | | | Determine | | MBio Ag/Ab Combo | | MBio/Ag/Ab Combo | |
|---|---|---|---|---|---|---|---|---|
| | | | Ag/Ab2 | | Standard Method | | VDSA Method | |
| Sample | Viral Load | | Ag | Ab | Ag | Ab | Ag | Ab |
| I.D. 1 | (Copies/ml) | EIA | s/co | s/co | s/co | s/co | s/co | s/co |
| 177-SL | 75,000 | NEG | NEG | NEG | 0.6 | −0.4 | 1.9 | 0.3 |
| 189-SL | 366,000 | NEG | NEG | NEG | 0.1 | 0.1 | 1.0 | 0.2 |
| 190-SL | 4,389,057 | NEG | POS | NEG | 1.7 | −0.1 | 12 | 0.5 |
| 191-SL | 9,627,991 | NEG | POS | NEG | 2.3 | 0.1 | 16 | 0.2 |
| 198-SL | 2,028 | NEG | NEG | NEG | 0.3 | −0.2 | 0.69 | NA |

1 Acute HIV-1 samples acquired from Antiviral Research Center, University of California, San Diego; La Jolla, CA.
2Alere Determine ™ HIV 1/2 Ag/Ab Combo assay

The results disclosed herein demonstrate the feasibility of incorporating a p24 Ag detection-enhancing viral disruption mechanism into a fourth-generation rapid HIV assay (i.e., an Ag/Ab combo assay that detects both HIV antigens and host antibodies against HIV antigens) without sacrificing HIV and co-infection serology components of the assay. The disclosed assays may be further improved by optimization of the VDSA reagent and by direct conjugation of fluorescent molecules to the detection antibody and antigen(s). This latter improvement would eliminate the need for the SA-SLP3 addition and incubation steps.

### Example 4

### Use of Zwitterionic Detergents for Disrupting HIV-1 Virions and for Improving p24 Antigen Detection

Zwitterionic detergents may be an alternative to the use of non-ionic or ionic detergents for the purpose of disrupting HIV-1 virions and for improving p24 antigen detection. Empigen BB is supplied (Sigma #45165) as a 35% aqueous solution; this reagent is diluted to a working concentration of 10% in H₂O. P24 antigen detection assays are initiated by combining 18 μl of sample (whole blood, plasma, or serum) with 2 μl 10% v/v Empigen BB (to yield 1% Empigen BB in sample) and mixing via simple aspiration. Following a 5 min incubation (time range of 1-15 minutes), 20 μl of sample dilution buffer [1× phosphate-buffered saline, pH 7.4 (Fisher Bioreagents #BP399-10), 0.4 mg/ml mouse IgG (Roche Custom Biotech, Indianapolis, Ind. #11200941103), 0.8 mg/ml poly-mouse IgG (Roche Custom Biotech #11816438103), 5 mg/ml bovine serum albumin (BSA), 0.05% Tween-20, 0.025% sodium azide, 3 nM biotinylated gp41 HIV antigen (Fitzgerald Industries International #30-AH26), and 45 nM biotinylated anti-p24 monoclonal antibody (US Biological #H6003-30A)] is added and the sample is mixed by an aspirate/dispense or vortexing method.

Note that the volume of added sample dilution buffer may range between 20 μl and 180 μl to yield a final concentration of between 0.5% and 0.1% Empigen BB. This assembled reaction volume is then added to the entrance port of an HIV Ag/Ab combo assay waveguide cartridge. During a 20-min incubation period at room temperature (5-60 min time range), sample p24 antigen becomes complexed with the biotinylated detect anti-p24 monoclonal antibody (mAb) and the immobilized capture anti-p24 mAb. During the same incubation period, anti-gp41 antibodies in the sample bridge biotinylated gp41 and immobilized capture gp41. Detection of the immobilized biotinylated complexes is achieved by adding 80 μl of 3 nM streptavidin-conjugated SureLight P3 (SA-SLP3) with incubation for an additional 15 min (time range=5 to 30 min) at room temperature. The waveguide array surface is washed by adding 200 μl (range=25-500 μl) of the following wash buffer to the cartridge entrance port: 200 mM NaCl, 2 mg/ml BSA, 0.2% v/v Tween-20, and 1×PBS, pH 7.4. Once the wash buffer has completed its exit from the entrance port, the waveguide is imaged on a fluorescence reader to analyze light signals emitted by the features (“spots”) printed onto the waveguide surface. The analytical device may be the waveguide-based device as described in U.S. patent application Ser. No. 13/233,794, which is hereby incorporated by reference into this disclosure.

For samples that do not originate in an EDTA-coated Vacutainer vial, EDTA may be included with the Empigen BB or may be added to yield 2-5 mM EDTA upon addition to the sample (the purpose of EDTA addition is explained in EXAMPLE 13). If the ratio of sample+Empigen to sample dilution buffer is greater than 1:1, the concentration of biotinylated anti-p24 detect mAb in the sample dilution buffer would likely be reduced to yield 20-25 nM detect mAb final in the fully assembled assay sample. Likewise, biotin-gp41 would be reduced to yield between 0.5 and 2.0 nM in the fully assembled sample (titration experiments would be performed to determine the optimal concentration). In this example, the detect mAb and gp41 are biotinylated. These detection agents (and others that may be included in the assay) may instead be directly labeled with fluorescent molecules (such as Alexa fluor-647, DyeLight-650, or SureLight P3), which would eliminate the need for the streptavidin-conjugated SureLight P3 addition step and the subsequent incubation period with this reagent.

### Example 5

### Blocking of CD59 Function to Enhance HIV-1 Virolysis

Assays are initiated by combining 21 μl of sample and 9 μl of sample dilution buffer comprised of 1× phosphate-buffered saline, pH 7.4 (Fisher Bioreagents #BP399-10), 0.67 mg/ml mouse IgG (Roche Custom Biotech, Indianapolis, Ind. #11200941103), 1.33 mg/ml poly-mouse IgG (Roche Custom Biotech #11816438103), 10 mg/ml bovine serum albumin (BSA), 0.33% Tween-20, 0.025% sodium azide, and 73.3 nM biotinylated anti-p24 monoclonal antibody (US Biological #H6003-30A), and 200 nM anti-CD59 antibody (range: 50-400 nM, to yield 15-120 nM final in the assembled reaction). The assembled assay reaction is incubated at room temperature (or 37 C) for 20-60 min to permit complement-mediated virolysis of HIV-1.

The assay reaction volume is then added to the entrance port of a waveguide cartridge containing a spatial array of capture agents, including anti-p24 capture antibody. During a 20-min incubation period at room temperature (5-60 min time range), sample p24 antigen becomes complexed with the biotinylated detect anti-p24 monoclonal antibody (mAb) and the immobilized capture anti-p24 mAb. Detection of the immobilized biotinylated complexes is achieved by adding 80 μl of 3 nM streptavidin-conjugated SureLight P3 (SA-SLP3) with incubation for an additional 15 min (time range=5 to 30 min) at room temperature. The waveguide array surface is washed by adding 200 μl (range=25-500 μl) of the following wash buffer to the cartridge entrance port: 200 mM NaCl, 2 mg/ml BSA, 0.2% v/v Tween-20, and 1×PBS, pH 7.4. Once the wash buffer has exited the entrance port, the waveguide is imaged on a fluorescence reader to analyze light signals emitted by the features (“spots”) printed onto the waveguide surface. The analytical device may be the waveguide based device as described in U.S. patent application Ser. No. 13/233,794, which is hereby incorporated by reference into this disclosure.

### Example 6

### Acid Disruption of Immune Complex

A patient sample of whole blood, plasma, or serum that is to be tested for the presence of target antigen is mixed with a low pH buffer (e.g. 50 mM Glycine-HCl pH 2.5) causing disruption of antibody-antigen complexes. After incubation, the sample mixture is neutralized to pH 6.5-7.5 by addition of 100 mM Phosphate assay buffer having pH 7.5. The neutralized sample is then subjected to a target identification/detection assay wherein freed antigen is detected. One example of such assay using waveguide is described in U.S. patent application Ser. No. 13/233,794, which is hereby incorporated by reference into this disclosure.

Alternatively, examples of other buffers or solutions that may be used include, glycine buffers in concentration 0.001M to 0.1M, at pH 2.0-3.5, Sodium Citrate buffer, Sodium Acetate buffer, Phosphate Citrate buffer. Non-buffered low pH solutions may also be used, which may include Hydrochloric Acid, Acetic Acid, Phosphoric Acid, or any other acid. Neutralizing solutions that may be used include but are not limited to Phosphate, Borate, Tris, MES, HEPES, or any other buffer in the 6.5-8 pH range. NaOH up to 0.1M may also be used for neutralization.

A model sample is made in which equimolar amounts of recombinant p24 antigen and monoclonal mouse anti-p24 antibody are mixed in PBS buffer at pH 7.2. The mixture is incubated 30 minutes at room temperature. A sample of the mixture is treated with an equal volume of 0.2M Glycine buffer. After 30 minutes incubation at room temperature, an equal volume of 0.5M sodium phosphate buffer pH 7.5 is added to return the overall pH to neutral. The sample is assayed for presence of p24 antigen using a sandwich type fluorescence immunoassay. When compared to a sample of the p24-antibody mixture that had not been subjected to Glycine treatment more signal is derived from the Glycine treated sample, indicating disruption of p24-antibody complexes caused by Glycine treatment prior to assay. The glycine solution may have a concentration of from 0.0.001 to 1M, having a pH in the range of 2-3.5.

In another example, serum from a suspected p24 containing sample is treated with an equal volume of 0.2M Glycine pH 2.5 buffer. After 30 minutes incubation at room temperature an equal volume of 0.5M sodium phosphate buffer pH 7.5 is added to return the overall pH to neutral. The sample is assayed for presence of p24 antigen using a sandwich type fluorescence immunoassay or a waveguide based assay as described in U.S. patent application Ser. No. 13/233,794, which is hereby incorporated by reference into this disclosure.

### Example 7

### Chaotrope Disruption of Antibody-Antigen Complexes

A patient sample of whole blood, plasma, or serum that is to be tested for the presence of target antigen is mixed with a concentrated chaotropic salt solution causing disruption of antibody-antigen complexes. After incubation, the sample mixture is neutralized by dilution of the chaotrope in assay buffer. The neutralized sample is then subjected to a target identification using a waveguide based assay as described in U.S. patent application Ser. No. 13/233,794, which is hereby incorporated by reference into this disclosure. Examples of chaotropic agents include but are not limited to butanol, ethanol, Guanidinium Chloride, Lithium perchlorate, Lithium Acetate, Magnesium Chloride, Phenol, Propanol, Sodium Dodecyl Sulfate, Thiourea, or Urea.

In another experiment, a sample suspected of containing p24 that is bound in immune complexes is mixed with equal volume of 4M urea and incubated for 30 minutes at room temperature. Following incubation, the sample is diluted with 9 volumes of 1×PBS buffer. The resulting sample solution is transferred to an analytical device, wherein detection of P24 antigen released by the lysis/disruption method is carried out. The analytical device may be the waveguide based device as described in U.S. patent application Ser. No. 13/233,794, which is hereby incorporated by reference into this disclosure.

In one specific experiment, a p24 antigen detection assay is initiated by combining 20 μl of sample (whole blood, plasma, or serum) with 20 μl of 4 M urea and thoroughly mixed. Following a 30-min incubation at room temperature, the 40 μl sample mixture is diluted into 360 μl (9 volumes) of a sample dilution buffer that includes labeled anti-p24 antibody and competitors of heterophilic antibodies [1× phosphate-buffered saline, pH 7.4 (Fisher Bioreagents #BP399-10), 0.1 mg/ml mouse IgG (Roche Custom Biotech, Indianapolis, Ind. #11200941103), 0.2 mg/ml poly-mouse IgG (Roche Custom Biotech #11816438103), 5 mg/ml bovine serum albumin (BSA), 0.05% Tween-20, 0.025% sodium azide, and 20 nM biotinylated anti-p24 monoclonal antibody (US Biological #H6003-30A)]. The assembled reaction volume is then added to the entrance port of a p24 antigen assay waveguide cartridge (waveguide spatial array includes anti-p24 antigen capture antibody features). During a 20-min incubation period at room temperature (5-60 min time range), p24 antigen in the sample becomes complexed with the biotinylated detect anti-p24 monoclonal antibody (mAb) and also with the immobilized capture anti-p24 mAb. Detection of the immobilized biotinylated complexes is achieved by adding 80 μl of 3 nM streptavidin-conjugated SureLight P3 (SA-SLP3; range=0.5-10 nM) with incubation for an additional 15 min (time range may be 1 to 30 min or longer) at room temperature. The waveguide array surface is washed by adding 200 μl (range may be 25-500 μl) of the following wash buffer to the cartridge entrance port: 200 mM NaCl, 2 mg/ml BSA, 0.2% v/v Tween-20, and 1×PBS, pH 7.4. Once the wash buffer has exited the entrance port, the waveguide is imaged on a fluorescence reader to analyze light signals emitted by the features (“spots”) printed onto the waveguide surface. The waveguide and the methods of detection are similar to those described in U.S. patent application Ser. No. 13/233,794, which is hereby incorporated by reference into this disclosure. Other detection methods can also be used here. For instance, directly fluor-labeled detection antibody, can be substituted for the biotin:SA-SLP3 method in the above protocol. This substitution would eliminate the SA-SLP3 addition and incubation steps.

### Example 8

### Heat Disruption of Antibody-Antigen Complex

A blood sample is collected from a suspected HIV patient by venipuncture or fingerstick. 50 μL of whole blood is transferred to a test tube. 50 μL of a 2× concentrated assay buffer such as 10 mM Na₃PO₄ pH 7, 0.05 tween 20, 1% BSA is added, and the sample is incubated at 95° C. for 20 minutes. The sample is then allowed to cool to room temperature and transferred to an waveguide-based device wherein detection of P24 antigen released by the lysis/disruption method is carried out. The waveguide and the methods of detection are similar to those described in U.S. patent application Ser. No. 13/233,794, which is hereby incorporated by reference into this disclosure.

### Example 9

### Combined Use of Heat and Detergent for Disruption of Antibody-Antigen Complex

Detergent is included primarily to limit protein aggregation. Detergent may help in both lysis of virions and disruption of Ag:Ab complexes. The combination of heat and detergent may result in irreversibly denatured antigens and/or antibodies (proteins in general). Denaturation of antigens may be a problem if immunoassay antibodies recognize a conformational antigen epitope (which may be lost by denaturation). Use of monoclonal antibodies that recognize the denatured antigen population may solve this problem.

In one specific experiment, a p24 antigen detection assay is initiated by combining 20 μl of sample (whole blood, plasma, or serum) with 5 μl of 5× heat shock buffer [1× phosphate-buffered saline, pH 7.4 (Fisher Bioreagents #BP399-10), 5.0% v/v Triton X-100, 2.5% w/v SDS] in an eppendorf tube. The diluted sample is mixed, then incubated at 85 C (preferred temperature range is 75-95 C, or 90-95 C) in a water bath or heat block (preferably with a heated lid for the purpose of reducing condensation) for 4 min. The tube is returned to room temperature and briefly spun in a microcentrifuge to combine the condensate with the solution at the bottom of the tube. A 50-μl volume of sample dilution buffer that includes labeled anti-p24 antibody and competitors of heterophilic antibodies 1× phosphate-buffered saline, pH 7.4 (Fisher Bioreagents #BP399-10), 0.3 mg/ml mouse IgG (Roche Custom Biotech, Indianapolis, Ind. #11200941103), 0.6 mg/ml poly-mouse IgG (Roche Custom Biotech #11816438103), 7.5 mg/ml bovine serum albumin (BSA), 0.15% Tween-20, 0.025% w/v sodium azide, and 30 nM biotinylated anti-p24 monoclonal antibody (US Biological #H6003-30A)] is added to the 25 μl heat-treated sample.

The assembled reaction volume is then added to the entrance port of a p24 antigen assay waveguide cartridge (waveguide spatial array includes anti-p24 antigen capture antibody features). During a 20-min incubation period at room temperature (5-60 min time range), sample p24 antigen becomes complexed with the biotinylated detect anti-p24 monoclonal antibody (mAb) and the immobilized capture anti-p24 mAb. Detection of the immobilized biotinylated complexes is achieved by adding 80 μl of 3 nM streptavidin-conjugated SureLight P3 (SA-SLP3) with incubation for an additional 15 min (time range=5 to 30 min) at room temperature. The waveguide array surface is washed by adding 200 μl (range=25-500 μl) of the following wash buffer to the cartridge entrance port: 200 mM NaCl, 2 mg/ml BSA, 0.2% v/v Tween-20, and 1×PBS, pH 7.4. Once the wash buffer has exited the entrance port, the waveguide is imaged on a fluorescence reader to analyze light signals emitted by the features (“spots”) printed onto the waveguide surface. The waveguide based device and the methods of detection are similar to those described in U.S. patent application Ser. No. 13/233,794, which is hereby incorporated by reference into this disclosure.

Other detection methods can also be used here. For instance, directly fluor-labeled detection antibody, can be substituted for the biotin:SA-SLP3 method in the above protocol. Also, the detergent components of the heat shock buffer may be replaced with (1) 5% Triton X-100 only, (2) 5% Empigen BB, or (3) 5% Triton only, and combinations of these and SDS or sodium deoxycholate may be used as well.

### Example 10

### Sonic Disruption of Antibody-Antigen Complex

A blood sample is collected from a suspected HIV patient by venipuncture or fingerstick. 50 uL of blood is transferred to a test tube. 50 μl of a 2× concentrated assay buffer such as 10 mM Na₃PO₄ pH 7, 0.05 tween 20, 1% BSA is added, and the sample test tube is immersed in a sonication vessel and subjected to high power sonication for about 10 minutes (range: 1-20 min). The sample is then transferred to a waveguide based analytical device, wherein detection of P24 antigen released by the lysis/disruption method is carried out. The waveguide based device and the methods of detection are similar to those described in U.S. patent application Ser. No. 13/233,794, which is hereby incorporated by reference into this disclosure.

### Example 11

### Use of Detergent for Disruption of Antibody-Antigen Complex

A patient sample of whole blood, plasma, or serum that is to be tested for the presence of target antigen is mixed with a concentrated detergent solution causing disruption of antibody-antigen complexes. After incubation, the sample mixture is neutralized by dilution of the detergent in assay buffer. The diluted sample is then subjected to a target identification and/or detection assay.

In one specific experiment, a blood sample is collected from a suspected HIV patient by venipuncture or fingerstick. 50 uL of blood is transferred to a test tube. 50 μl of a 2× concentrated detergent buffer is added (for example, 2×PBS, 5% Triton X100), and the sample is incubated at room temperature for 20 minutes. The sample is then transferred to a waveguide based analytical device, wherein detection of P24 antigen released by the disruption method is carried out. The waveguide based device and the methods of detection are similar to those described in U.S. patent application Ser. No. 13/233,794, which is hereby incorporated by reference into this disclosure.

### Example 12

### Combination of Disruption Methods

A patient sample of whole blood, plasma, or serum that is to be tested for the presence of target antigen is mixed with a low pH buffer (e.g. 50 mM Glycine-HCl pH 2.5), and subjected to 95 C incubation for 20 minutes, causing disruption of antibody-antigen complexes. After incubation, the sample mixture is cooled to room temperature neutralized to pH 6.5-7.5 by addition of 100 mM Phosphate assay buffer pH 7.5. The neutralized sample is then subjected to a target identification/detection assay by using a waveguide based device wherein freed antigen is detected. The waveguide based device and the methods of detection are similar to those described in U.S. patent application Ser. No. 13/233,794, which is hereby incorporated by reference into this disclosure.

### Example 13

### Release and Detection of Immune Complexes Bound to Red Blood Cells in a Patient Sample

Besides immune complexes that are circulating in blood, some antibody-antigen immune complexes are bound to red blood cells (RBC). Beck, Z., et al., Human Erythrocytes selectively bind and enrich infectious HIV-1 virions. PLoS One 4: e8297 (2009). Therefore releasing immune complexes from red blood cells prior to or in combination with other immune complex disruption methods described above may release antigen from RBCs and therefore increase the total amount of detectable antigens. HIV-1 readily binds to the surface of erythrocytes (RBC-associated HIV-1 is approximately 100-fold more efficient, via trans infection, than free virus for infection of CD4(+) cells).

Essentially all of the RBC-bound HIV-1 is released by treatment with EDTA. When blood samples are received in vacutainer vials coated with EDTA, RBC-bound HIV-1 is released by EDTA induced RBC lysis. However, when assaying drops of blood from finger sticks where EDTA is not included in the blood collection device, EDTA (e.g., 5-20 mM) may be included in the sample dilution buffer used for whole blood HIV-1 antigen detection assays.

Use of EDTA blood collection tubes may cause disruption of the interaction between immune complexes and red blood cells, releasing immune complexes into solution, where any or all of the previously discussed techniques can be used to release antigen from the complex.

### Example 14

### Pretreatment of a Sample to Release Analyte from Circulating Immune Complexes and/or Intact Virus Particles in a Whole Blood Sample

A blood sample suspected of containing target analyte that is either in complex with circulating immune complexes, or still contained within viral particles, or contained in both, is obtained. 100 μl of the whole blood is transferred to a reaction vessel, such as a 1.5 mL test tube. The sample is diluted with an equal volume of a lysis/disruption buffer containing 0.1 M Glycine pH 2.5, 1% TritonX-100, 1% Sodium deoxycholate and incubated at 90 C for 5 minutes, causing disassociation of circulating immune complexes and disruption of virus particles, resulting in release of target antigen into the sample matrix. The sample is cooled to room temperature and 1 equal volume of 0.2M Sodium Phosphate pH 8 is added, neutralizing the Glycine and diluting the detergents. The sample is then analyzed on a waveguide-based device, and the presence/absence and quantity of target analyte is determined. The waveguide-based device and the methods of detection are similar to those described in U.S. patent application Ser. No. 13/233,794, which is hereby incorporated by reference into this disclosure.

### Example 15

### Methods of Viral/Immune Complex Disruption without Neutralization: Disrupt—Detect

A volume of patient blood is collected by venipuncture, and mixed with an equal volume of a 2× concentrated lysis/disruption buffer. The sample is mixed and incubated for a period of time at a certain temperature. The sample is then transferred to an analytical device to test for the presence of certain biomarkers indicative of the presence of an infection.

In one specific experiment, a blood sample is collected from a patient by venipuncture or fingerstick. 50 uL of blood is transferred to a test tube. 50 μl of a 2× concentrated lysis/disruption buffer is added, and the sample is incubated at room temperature for 20 minutes. The sample is then transferred to an analytical device such as a waveguide based device, wherein detection of P24 antigen released by the lysis/disruption method is carried out. The waveguide based device and the methods of detection are similar to those described in U.S. patent application Ser. No. 13/233,794, which is hereby incorporated by reference into this disclosure. The concentrated lysis/disruption buffer may be any buffer disclosed herein or combination thereof. By way of example, 2× concentrated lysis/disruption buffer may be 0.2M Glycine-HCl pH 2.5.

### Example 16

### Methods of Viral/Immune Complex Disruption with Neutralization Step: Disrupt—Neutralize-Detect

A volume of patient blood is collected by venipuncture, and mixed with an equal volume of a 2× concentrated lysis/disruption buffer. The sample is mixed, and incubated for a period of time at a certain temperature. The sample is then mixed to an equal volume of a 2× concentrated neutralization buffer to neutralize the effects of the lysis/disruption buffer. The sample is then transferred to an analytical device to test for the presence of certain biomarkers indicative of the presence of an infection.

### Example 17

### Methods of Viral/Immune Complex Disruption with Neutralization and Concentrations Steps: Disrupt-Neutralize-Concentrate-Detect

A volume of patient blood is collected by venipuncture, and mixed with an equal volume of a 2× concentrated lysis/disruption buffer. The sample is mixed, and incubated for a period of time at a certain temperature. The sample is then mixed to an equal volume of a 2× concentrated neutralization buffer to neutralize the effects of the lysis/disruption buffer. The sample is then concentrated to a smaller volume by using a concentration technique. A suitable concentration technique would be, for example, a disposable centrifugal device that passes a portion of the sample solution through a molecular weight cut-off filter; the filter retains the molecules to be detected. The retained sample is then transferred to an analytical device to test for the presence of certain biomarkers indicative of the presence of an infection.

Changes may be made in the above methods and systems without departing from the scope hereof. It should thus be noted that the matter contained in the above description or shown in the accompanying drawings should be interpreted as illustrative and not in a limiting sense. The following claims are intended to cover generic and specific features described herein, as well as statements of the scope of the present method and system, which, as a matter of language, might be said to fall therebetween.

Although each of the embodiments have been illustrated with various components having particular respective orientations, it should be understood that the system and methods as described in the present disclosure may take on a variety of specific configurations or modifications with the various compositions being modified or substituted and still remain within the spirit and scope of the present disclosure. Furthermore, suitable equivalents may be used in place of or in addition to the various components or compositions, the function and use of such substitute or additional components being held to be familiar to those skilled in the art and are therefore regarded as falling within the scope of the present disclosure. Therefore, the present examples are to be considered as illustrative and not restrictive, and the present disclosure is not to be limited to the details given herein but may be modified within the scope of the appended claims.

### LIST OF REFERENCES

The following references, patents and publication of patent applications are either cited in this disclosure or are of relevance to the present disclosure. All documents listed below, along with other papers, patents and publication of patent applications cited throughout this disclosures, are hereby incorporated by reference as if the full contents are reproduced herein:
1. Layne, S. P., M. J. Merges, M. Dembo, J. L. Spouge, S. R. Conley, J. P. Moore, J. L. Raina, H. Renz, H. R. Gelderblom, and P. L. Nara. 1992. Factors underlying spontaneous inactivation and susceptibility to neutralization of human immunodeficiency virus. Virology 189: 695-714.
2. Summers, M. F., L. E. Henderson, M. R. Chance, J. W. Bess, Jr., T. L. South, P. R. Blake, I.

Sagi, G. Perez-Alvarado, R. C. Sowder III, et al. 1992. Nucleocapsid zinc fingers detected in retroviruses: EXAFS studies of intact viruses and the solution-state structure of the nucleocapsid protein from HIV-1. Protein Sci. 1: 563-574.
3. Nishanian, P., K. R. Huskins, S. Stehn, R. Detels, and J. L. Fahey. 1990. A simple method for improved assay demonstrates that HIV p24 antigen is present as immune complexes in most sera from HIV-infected individuals. J. Infectious Diseases 162: 21-28.
4. Schupbach, J., M. Flepp, D. Pontelli, Z. Tomasik, R. Luthy, and J. Boni. 1996. Heat-mediated immune complex dissociation and enzyme-linked immunosorbent assay signal amplification render p24 antigen detection in plasma as sensitive as HIV-1 RNA detection by polymerase chain reaction. AIDS 10: 1085-90.
5. Schupbach, J., Z. Tomasik, M. Knuchel, M. Opravil, H. F. Gunthard, D. Nadal, J. Boni, and the Swiss HIV Cohort Study (SHCS) and the Swiss HIV Mother+Child Cohort Study (MoCHiV). 2006. J. Medical Virology 78: 1003-1010.
6. Parpia, Z. A., R. Elghanian, A. Nabatiyan, D. R. Hardie, and D. M. Kelso. 2010. P24 antigen rapid test for diagnosis of acute pediatric HIV infection. J. Acquir. Immune Defic. Syndr. 55(4): 413-419.
7. Karris, M.; C. Anderson, S. Morris, D. Smith, and S. Little, 2012. Cost Savings Associated with Testing of Antibodies, Antigens, and Nucleic Acids for Diagnosis of Acute HIV Infection. J. Clin. Microbiol. 50: 1874-1878.
8. Nabitayan A, Parpia Z A, Elghanion R, Kelso D M. 2011. Membrane-based plasma collection device for point-of-care diagnosis of HIV. J Virol Methods 2011 April; 173(1):37-42.
9. Steinmetzer, K.; T. Seidel, A. Stallmach and E. Ermantraut, 2010. HIV Load Testing with Small Samples of Whole Blood. J. Clin. Microbiol. 48: 2786-2792.
10. Garcia, M.; M. Farias, M. Avila, and R. Rabinovich, 2012. Presence of p24-Antigen Associated to Erythrocyte in HIV-Positive Individuals Even in Patients with Undetectable Plasma Viral Load. PLoS ONE 6(1): e14544.
11. Schupbach, J. 2003. Viral RNA and p24 antigen as markers of HIV disease and antiretroviral treatment success. Int. Arch. Allergy Immunol. 132: 196-209.
12. Fiscus, S. A., C. D. Pilcher, W. C. Miller, K. A. Powers, I. F. Hoffman, M. Price, D. A. Chilongozi, C. Mapanje, R. Krysiak, S. Gama, F. E. A. Martinson, M. S. Cohen, and the Malawi-University of North Carolina Project Acute HIV Infection Study Team. 2007. Rapid, real-time detection of acute HIV infection in patients in Africa. J. Infectious Diseases 195: 416-24.
13. Popova, O. Ya., and L. S. Kositskaya. 1977. Dissociation of immune complexes and inactivation of bound antibodies by reducing agents. Immunochemistry 14(8): 633-635.
14. Brighenti, S., and Lerm, M (2012). How Mycobacterium tuberculosis Manipulates Innate and Adaptive Immunity How Mycobacterium tuberculosis Manipulates Innate and Adaptivetibodies by reducing agents. Imm Tuberculosis Pathogenicity, Dr. Pere-Joan Cardona (Ed.), ISBN: 978-953-307-942-4.
15. Vankayalapati, R., Barnes, P., (2009), Innate and adaptive immune responses to human Mycobacterium tuberculosis infection. Tuberculosis 89, 51, 577-580.
16. Raja, A., Ranganathan, U., Bethunaicken, R., (2006), Clinical value of specific detection of immune complex-bound antibodies in pulmonary tuberculosis. Diagnostic Microbiology and Infectious Disease, 56: 281-287.
17. Johnson, N., McNicol, M., Burton-Kee, E., Mowbray, J. (1981) Circulating Immune Complexes in tuberculosis. Thorax, 36:610-617.
18. Samuel, A., Ashtekar, M., Ganatra, R., (1984) Significance of circulating immune complexes in pulmonary tuberculosis. Clin. Exp. Immunol. 58; 317-324.
19. Beck, Z., et al. 2009. Human Erythrocytes selectively bind and enrich infectious HIV-1 virions. PLoS One 4: e8297.
20. Yu, Q., S. Cecilia, B. Shiramizu, and N. Hu. 2009, Virolysis of complement-resistant HIV-1 by antibodies in the plasmas from HIV-1-infected individuals. The Journal of Immunology 182: 128.10.
21. Hu, W. et al. 2010. A high affinity inhibitor of human CD59 enhances complement-mediated virolysis of HIV-1: implications for treatment of HIV-1/AIDS. J. Immunol. 184: 359-368.
22. Arora, A., JP Wali, P Seth, J S Guleria, P Aggarwal. 1991. Circulating Immune Complexes in Tuberculosis. Singapore Med J, 32, 116-118.

1. A method for determining the level of one or more biomarkers in a sample, the method comprising:
a) contacting the sample with a composition to form a sample mixture, wherein the composition comprises an ionic detergent, a nonionic detergent, and a salt,
b) loading the sample mixture into a device comprising a waveguide, allowing the one or more biomarker to bind to one or more capture molecules immobilized on the waveguide,
c) adding one or more labeling molecules into the device, allowing the labeling molecules to bind to their respective biomarkers, and
d) measuring the signal intensity emitted from the labeling molecules that are bound to the immobilized biomarkers and capture molecules on the waveguide to determine the level of the one or more biomarkers in the sample.

2. The method of claim 1, wherein the sample is a member selected from the group consisting of whole blood sample, serum, plasma and saliva.

3. The method of claim 1, further comprising a step of raising temperature of said sample mixture to at least 70° C. after step (a) but before step (b).

4. The method according to claim 1, wherein the sample comprises a plurality of biomarkers comprising at least one antigen originated from a pathogen and at least one antibody against the pathogen, and wherein the device comprises a plurality of capture molecules, at least one group of capture molecules being capable of capturing said at least one antigen, and at least one other group of capture molecules being capable of capturing said at least one antibody.

5. The method according to claim 1, wherein the composition further comprises an anti-CD59 antibody.

6. The method according to claim 1, wherein the composition has a pH of lower than 3.5.

7. The method of claim 6, further comprising a neutralizing step after step (a) but before step (b).

8. The method according to claim 1, wherein the sample is a whole blood sample.

9. The method according to claim 1, wherein the composition comprises Triton® X-100 at a concentration of 2-3% (v/v), sodium deoxycholate at a concentration of 2-3% (w/v), sodium dodecyl sulfate (SDS) at a concentration of 0.3-0.8% (w/v), NaCl at a concentration of 0.5-1M, EDTA at a concentration of 10-25 mM, and Tris-CI, pH 7.4, at a concentration of 30-80 mM.

10. The method according to claim 1, wherein the sample is derived from a blood sample, wherein the one or more biomarkers comprise a protein originating from the human immunodeficiency virus (HIV), and said method being capable of producing a statistically significant positive signal from a sample having an HIV viral load of 300,000 copies/ml or lower.

11. A composition for processing a sample to determine the level of a biomarker in the sample, said composition comprising
a) an ionic detergent, wherein the ionic detergent comprises deoxycholate;
b) a nonionic detergent, and
c) a salt.

12. The composition of claim 11, wherein the ionic detergent further comprises sodium dodecyl sulfate (SDS), wherein the SDS is present in the composition at a concentration of 0.1-1.5% (w/v).

13. The composition of claim 11, wherein the deoxycholate is sodium deoxycholate, and the sodium deoxycholate is present in the composition at a concentration of 1% to 5% (w/v)

14. The composition of claim 11, wherein the nonionic detergent comprises Triton® X-100, said Triton® X-100 being present in the composition at a concentration of 1-5% (v/v).

15. The composition of claim 11, wherein the composition is mixed with the sample at a certain ratio to form a sample mixture, wherein the concentration of the SDS in the sample mixture is in the range of 0.01-0.3% (w/v).

16. The composition of claim 11, wherein the deoxycholate is sodium deoxycholate, and wherein the composition is mixed with the sample at a certain ratio to form a sample mixture, the concentration of the sodium deoxycholate in the sample mixture being in the range of 0.1-1% (w/v).

17. The composition of claim 11, wherein the nonionic detergent comprises Triton® X-100, and wherein the composition is mixed with the sample at a certain ratio to form a sample mixture, the concentration of the Triton® X-100 in the sample mixture being in the range of 0.1-1% (v/v).

18. The composition of claim 11, further comprising an anti-CD59 antibody.

19. The composition of claim 11, wherein the composition comprises Triton® X-100 at a concentration of 2-3% (v/v), sodium deoxycholate at a concentration of 2-3% (w/v), sodium dodecyl sulfate (SDS) at a concentration of 0.3-0.8% (w/v), NaCl at a concentration of 0.5-1M, EDTA at a concentration of 10-25 mM, and Tris-CI, pH 7.4, at a concentration of 30-80 mM.

20. The composition of claim 11, wherein the composition comprises Triton® X-100 at a concentration of about 2.5% (v/v), sodium deoxycholate at a concentration of about 2.5% (w/v), sodium dodecyl sulfate (SDS) at a concentration of about 0.5% (w/v), NaCl at a concentration of about 0.75 M, EDTA at a concentration of about 17 mM, and Tris-CI, pH 7.4, at a concentration of about 50 mM.

21. (canceled)

22. A method for determining the level of one or more biomarkers in a sample, the method comprising:
a) contacting the sample with a composition to form a sample mixture, wherein the composition comprises an ionic detergent, a nonionic detergent, a salt, and one or more labeling molecules that bind to said one or more biomarkers,
b) loading the sample mixture into a device comprising a waveguide, allowing the one or more biomarkers to bind to one or more capture molecules immobilized on the waveguide, and
c) measuring the signal intensity emitted from the labeling molecules that are bound to the immobilized biomarkers and capture molecules on the waveguide to determine the level of the one or more biomarkers in the sample.
